(19) United States
(12) Patent Application Publication
(10) Pub. No.: US 2021/0187236 A1
(43) Pub. Date: Jun. 24, 2021

(54) METHOD FOR EXECUTING A SESSION OF DIFFUSION OF AN ACTIVE GASEOUS COMPOSITION IN A HABITABLE ENVIRONMENT

(71) Applicant: Evergreen Land Limited, Central Hong Kong (HK)

(72) Inventor: Olivier Partrat, Hong Kong (HK)

(21) Appl. No.: 16/762,591

(22) PCT Filed: Nov. 13, 2018
(86) PCT No.: PCT/IB2018/058890

§ 371 Date: May 8, 2020

(30) Foreign Application Priority Data
Nov. 15, 2017 (FR) 17/60762

(51) Int. Cl. A61M 21/02; G16H 40/67; F24F 11/64

(52) U.S. Cl. CPC A61M 21/02; A61M 2021/0016; F24F 11/64; G16H 40/67

(57) ABSTRACT

The invention relates to a method providing a diffusion procedure where diffusion is controlled in order to confer a diffusion desired for the composition, and, before the execution of the diffusion procedure, a previous procedure of treatment of the air of the environment occurs. The previous treatment procedure measures at least one physico-chemical parameter of said air; compares the measured parameter to a threshold; and, if the threshold is exceeded by the measured parameter, activates a device for treatment of the air that is arranged in order to modify the parameter in compliance with the threshold.

### CROSS-REFERENCE TO RELATED APPLICATION

This application claims the benefit of International application number PCT/162018/058890, filed Nov. 13, 2018 and French application number 1760762, filed Nov. 15, 2017 the entire contents of which are incorporated herein by reference.

### TECHNICAL FIELD

The invention relates to a method for executing a session of diffusion of an active gaseous composition in a habitable environment, as well as a system for the implementation of such a method.

It applies in particular to a session of diffusion of a gaseous composition, the active principle of which comprises at least one volatilised compound, for example at least one essential oil, in particular for aromatherapy purposes.

### BACKGROUND

The document FR-3 040 596 describes a device that diffuses an odoriferous gaseous composition, comprising a generator of a flow of said composition contained in a tank. In particular, this device can be controlled in order to confer the diffusion desired for said composition, in particular by varying the speed of flow of the flow of air in the tank.

However, in order for a session to be truly beneficial, in particular in the context of aromatherapy, it must be executed in a controlled environment, in particular with regard to the relative absence of pollutants in the air during said session.

Thus, above concentration of organic or inorganic volatile compound, and/or of particles in suspension in the air of the environment, a diffusion of an aromatherapeutic composition can turn out to be useless or even dangerous. Likewise, the effectiveness of the session is improved when the concentration of active composition in the air to be breathed is maintained in a defined therapeutic range.

The document WO-2017/186606 describes a method for generating a scented atmosphere in a room comprising a device for purifying air capable of eliminating a polluting substance, which intends to monitor the concentration of the polluting substance and diffuse at least one scented substance in said room when the monitored concentration falls below a predefined threshold.

In particular, in order to limit the risks of a potentially dangerous chemical reaction between the polluting and scented substances, this document intends to monitor the concentration of the polluting substance in order to control the diffusion of the scented substance.

However, this control is not compatible with the diffusion sessions envisaged, in particular in that they must be controlled according to the desired effects and not the concentration of pollutants.

### SUMMARY OF THE INVENTION

The invention aims to improve the prior art by proposing in particular a method for executing a session of diffusion of an active gaseous composition in a habitable environment, wherein the benefit of said session is optimised, in particular in relation to an aromatherapy session.

For this purpose, according to a first aspect, the invention proposes a method for executing a session of diffusion of an active gaseous composition in a habitable environment, said method providing a diffusion procedure during which at least one characteristic of said diffusion is controlled in order to confer a diffusion desired for said composition, said method intending, before the execution of said diffusion procedure, to execute a previous procedure of treatment of the air of the environment, said previous treatment procedure intending to:
measure at least one physico-chemical parameter of said air;
compare said measured parameter to a threshold; and,
if said threshold is exceeded by said measured parameter, activate a device for treatment of the air that is arranged in order to modify said parameter in compliance with said threshold.

According to a second aspect, the invention proposes a system for a session of diffusion of an active gaseous composition in a habitable environment, said system comprising a device for diffusion of said gaseous composition, a device for measuring at least one physico-chemical parameter of the air and a device for treating said air, said system further comprising an application for controlling said devices that is arranged for the implementation of such a method.

### BRIEF DESCRIPTION OF THE DRAWINGS

Other particularities and advantages of the invention will be clear in the following description, made in reference to the appended figures, in which:

FIG. 1 schematically shows the procedures implemented in an execution method according to an embodiment of the invention;

FIG. 2 schematically shows a system according to an embodiment of the invention.

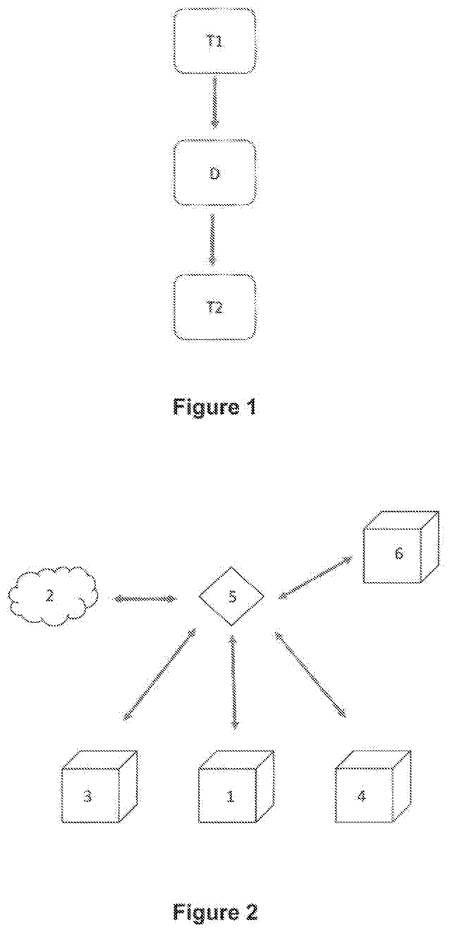

### DETAILED DESCRIPTION

In relation to these figures, a method for executing a session of diffusion of an active gaseous composition in a habitable environment, as well as a system for the implementation of such a session, are described below.

Advantageously, the invention can be implemented in a living environment, for example a room in particular having a volume of air of less than 200 m³, for example less than 80 m³. According to another intended use, the habitable environment can have a smaller volume of air, for example being the passenger compartment of a motor vehicle.

In particular, the session allows the diffusion of a gaseous composition, the active principle of which comprises at least one volatilised compound, for example at least one essential oil, in particular for aromatherapy purposes, said composition thus being breathed in by the occupant(s) of the environment in order to confer the expected effect, for example by contributing to reducing oxidation and inflammations. According to other envisioned uses, the action of the gaseous composition can confer at least one other well-being function, for example such as perfuming, deodorising, biocidal, bactericidal, fungicidal, repulsive or attractive functions.

To do this, the system comprises a device 1 for diffusion of the active gaseous composition. According to an advantageous embodiment, the diffusion is called dry in that it is obtained by a flow of air at a formulation of the composition to be diffused that can be volatilised.

For example, specific formulations are described below according to the effect desired for the session.

Formulation 1: cerebral energy, memory function

| Essential | Percentages in the |
|---|---|
| Oils/compounds | composition |
| Ravintsara | 0 to 5.0% |
| Ylang-ylang | 0 to 5.0% |
| Lemon | 10% to 30% |
| Eucalyptus | 10% to 30% |
| Peppermint | 10% to 30% |
| Rosemary | 0 to 10% |
| Vanillin | 0 to 10% |

Formulation 2: relaxing function

| Essential | Percentages in the |
|---|---|
| Oils/compounds | composition |
| Lavender | 0 to 10% |
| Roman Chamomile | 0 to 10% |
| Ylan Tlang | 0 to 10% |
| Marjoram | 0 to 10% |
| Lemon | 10% to 30% |
| Bergamot | 10% to 30% |
| Mandarin | 10% to 30% |
| Sweet Orange | 10% to 30% |
| Bigarade | 0 to 10% |
| Shiu | 0 to 10% |
| Vanillin | 0 to 10% |

Formulation 3: sleep improvement function

| Essential | Percentages in the |
|---|---|
| oils/compounds | composition |
| Lavender | 30% to 50% |
| Roman Chamomile | 0 to 10% |
| Marjoram | 0 to 10% |
| Sweet Orange | 30% to 50% |
| Bigarade | 0 to 10% |
| Vanillin | 0 to 10% |

Formulation 4: respiratory function

| Essential | Percentages in the |
|---|---|
| oils/compounds | composition |
| Eucalyptus | 30% to 50% |
| Mentha arvensis | 10% to 30% |
| Ravintsara | 0 to 10% |
| Lavender | 0 to 10% |
| Niaouli | 30% to 50% |
| Cypress | 0 to 10% |
| Vanillin | 0 to 10% |

Formulation 5: anti-mosquito repulsive function

| Essential | Percentages in the |
|---|---|
| oils/compounds | composition |
| Eucalyptus | 10% to 30% |
| Geraniol | 30% to 50% |
| Lavender | 30% to 50% |
| Mint | 0 to 10% |
| Vanillin | 0 to 10% |

According to one embodiment, in particular known from the document FR-3 040 596, the diffusion device 1 comprises at least one cartridge containing a formulation of the composition to be diffused that can be volatilised, and means for generating a flow of air at said cartridge. Alternatively, optionally in addition to the means for volatilisation via flow of air, the diffusion device 1 comprises means for thermal and/or vibrational volatilisation, for example piezoelectric, of the composition to be diffused.

Advantageously, the cartridge can be removable and/or resealable between two diffusion procedures. Moreover, the diffusion device 1 can comprise at least two cartridges that can be used together or separately by the volatilisation means, in order to envision a combined or separate action of the formulations that can be volatilised contained in each of the cartridges.

The diffusion session comprises a procedure of diffusion D during which at least one characteristic of the diffusion is controlled in order to confer a diffusion desired for the composition, in particular by projecting to control the volatilisation means. In particular, the characteristic of control of the diffusion can be chosen from the diffusion time and the concentration of gaseous composition of a diffused flow of air.

The system for executing the session comprises an application 2 for controlling the diffusion device 1, in particular the speed of generation of the flow of air by the volatilisation means of the device. The application 2 can be implanted in a digital medium, for example integrated into the diffusion device 1 or formed by an electronic apparatus present in the environment, such as a computer or a smartphone.

The digital medium comprises means for communication, for example of the Bluetooth or Wi-Fi type, with the diffusion device 1, as well as an interface of use. Moreover, the digital medium comprises memory means, in particular for the storage of the history of use and of the relevant data for the sessions to be executed.

In relation to FIG. 2, the system for executing the session comprises a module 5 dedicated to the control of the devices 1, 3, 4, wherein the module can be permanently positioned in the environment in order to receive the control instructions from the application 2 in order to be able providing the control, in particular autonomously in the absence of the digital medium. In particular, the module 5 comprises means for communication with, one the one hand the application 2 and, on the other hand, the devices 1, 3, 4, the application thus being able to no longer have to communicate directly with the devices.

According to one embodiment, the diffusion procedure D provides an initial determination of at least one characteristic of control of the diffusion, the determination being carried out according to the diffusion session to be executed and/or the nature of the environment. In particular, the characteristics of control can be the diffusion time, for example approximately 20 minutes, and the speed of the flow of air, wherein the diffusion can nevertheless be interrupted and/or extended or modulated manually by the user.

The cartridge containing the formulation that can be volatilised to be diffused contains information, in particular regarding the nature of the formulation that it contains, and the system comprises means for reading the information that are capable of transmitting the information to the application 2 in order to control at least one characteristic of the diffusion on the basis of the information. According to one embodiment, the information is contained in a medium of the RFID type.

Thus, when a cartridge is inserted into the diffusion device 1, the diffusion procedure D can be automatically controlled by the application 2 according to the session to be executed.

In particular, the volatilisation means are controlled in order to adapt the speed and/or the nature of the flow of air according to the expected concentration of active gaseous composition. Indeed, a faster and/or more concentrated flow allows to increase the concentration of the gaseous composition in the air of the environment.

The system comprises a device 3 for measuring at least one physico-chemical parameter of the air, for example chosen from a concentration of organic or inorganic volatile component, a concentration of particles in suspension, the humidity, the temperature and the pressure. In particular, such a device 3 comprises at least one sensor of the parameter and means for measuring and communicating the measured parameter.

Via this device 3, the diffusion procedure D provides for an iterative measurement of at least one physico-chemical parameter of the air of the environment, at least one characteristic of the diffusion being controlled according to the measured parameter.

The device 3 can thus measure a parameter linked to the gaseous composition diffused, for example a concentration of at least one organic volatile compound contained in the composition, the device comprising means for communicating the parameter to the application 2 which, after comparison to a desired concentration, controls the diffusion device 1 dynamically. Thus, the effectiveness of the session is improved since the concentration of active composition in the air can be maintained in a defined therapeutic range.

The system also comprises a device 4 for treating the air of the environment, for example in the form of an air purifier that can in particular reduce concentrations of organic or inorganic volatile component, concentrations of particles in suspension, in particular by filtration.

Via such a device 4 and before the execution of the diffusion procedure D, the method intends to execute a previous procedure of treatment T1 of the air of the environment, the previous treatment procedure intending to:
measure at least one physico-chemical parameter of the air;
compare the measured parameter to a threshold; and,
if the threshold is exceeded by the measured parameter, activate the device 4 for treatment of the air which is arranged to modify the parameter in compliance with the threshold.

The devices 1, 4, 5 of the system can be physically separate by communicating with the application 2 wirelessly, or via at least one wired link. According to another embodiment, at least two of the devices 1, 4, 5 can be integrated into the same apparatus.

According to one embodiment, the device 3 can measure a parameter related to the pollution of the air of the environment, for example concentrations of organic or inorganic volatile component, concentrations of particles in suspension, in particular according to the size of the particles, but also of the environment itself, for example its humidity, its temperature and its pressure.

The measured parameter is thus communicated to the application 2 which, after comparison to the threshold, can interact with the treatment device 4 in order to be able to control it. In particular, according to the session to be executed, the application 2 integrates the parameter(s) and their respective threshold, the application interacting with the measurement device 3 in order to obtain the relevant data.

Thus, by setting at least one optimal threshold for at least one of the physico-chemical parameters of the air of the environment, the application 2 can control the diffusion procedure D only after it has controlled the device 4 for treatment of the air, in particular with regard to the relative absence of pollutants in the air during the session.

In particular, the air breathed during the session is thus healthy in that it satisfies a given quality, wherein the quality can further be improved by providing for other types of parameter for control of the session, in particular relative to the light in the environment. According to one embodiment, a light sensor can be used to control a light emitter 6 in accordance with the session to be executed, in order in particular to be able to combine the benefits of light therapy with those of the diffusion of the gaseous composition.

According to one embodiment, the method intends to activate the device 4 for treatment of the air for a given time and/or execute the diffusion procedure D after the passage of a given time, in particular approximately 15 minutes, after the previous treatment procedure T1. In particular, the treatment procedure T1 and the diffusion procedure D are carried out independently, one after the other, in order to be able to each be optimised for their goal.

In order to make the carrying out of the diffusion procedure D in an optimal environment reliable, the method provides a measurement of at least one physico-chemical parameter of the air after the previous treatment procedure T1 and, if a threshold is exceeded by the measured parameter, execution of an additional procedure of treatment of the air before activation of the diffusion device 1.

Besides this pre-treatment of the air, advantageously, the method provides, after the execution of the diffusion procedure D, a later procedure of treatment T2 of the air of the environment during which the device 4 for treatment of the air is activated if necessary in order to modify at least one physico-chemical parameter of the air.

In particular, the modified parameter is related to the gaseous composition diffused, in particular by treating the air after the diffusion in order for the concentration of gaseous composition to be reduced in order to truly put an end to the session in the environment.

Advantageously, the application 2 can provide the user with at least one of the following functions:
REMOTE CONTROL: access and trigger the devices 1, 3, 4 from anywhere;
CONTROL THE DIFFUSION: start or program a session with 7-day programming;
MONITOR THE QUALITY OF THE AIR: know what you are breathing with a single glance, for example on your smartphone;
MONITOR YOUR AIR PURIFICATION: monitor the purification activity of the treatment device 4 in real time;
QUICKLY VIEW THE REPORTS: easily access detailed reports on the history of the quality of the air and export them to your desktop or your laptop computer;
VERIFY YOUR LIFE CONSUMABLES: see how much time remains for your capsules and your filter of the treatment device 4 before they have to be changed.

What is claimed is:

1. A method for executing a session of diffusion of an active gaseous composition in a habitable environment, the method providing a diffusion procedure during which at least one characteristic of the diffusion is controlled in order to confer a diffusion desired for the composition, the method being before the execution of the diffusion procedure, to execute a previous procedure of treatment of the air of the environment, the previous treatment procedure:
measures at least one physico-chemical parameter of the air;
compares the measured parameter to a threshold; and,
if the threshold is exceeded by the measured parameter, activates a device for treatment of the air that is arranged in order to modify the parameter in compliance with the threshold.

2. The execution method according to claim 1, wherein it provides, after the execution of the diffusion procedure, a later second procedure of treatment of the air of the environment during which a device for treatment of the air is activated in order to modify at least one physico-chemical parameter of the air.

3. The execution method according to claim 2, wherein the modified parameter is related to the gaseous composition diffused.

4. The execution method according to claim 1, wherein diffusion procedure provides for an iterative measurement of at least one physico-chemical parameter of the air of the environment, at least one characteristic of the diffusion being controlled according to the measured parameter.

5. The execution method according to claim 4, wherein the parameter measured is related to the gaseous composition diffused.

6. The execution method according to claim 1, wherein the diffusion procedure provides for an initial determination of at least one characteristic of control of the diffusion, the determination being carried out on the basis of the diffusion session to be executed and/or the nature of the environment.

7. The execution method according to claim 1, wherein the characteristic of control of the diffusion is chosen from the diffusion time and the concentration of gaseous composition in a diffused flow of air.

8. The execution method according to claim 1, wherein the diffusion procedure intends to includes control means for volatilisation of the composition to be diffused.

9. The execution method according to claim 7, wherein the volatilisation means are controlled in order to adapt the speed and/or the nature of the flow of air according to the expected concentration of gaseous composition.

10. The execution method according to claim 1, wherein the physico-chemical parameter is chosen from concentrations of organic or inorganic volatile component and concentrations of particles in suspension.

11. The execution method according to claim 1, wherein in that it intends to activate the device for treatment of the air for a given time.

12. The execution method according to claim 1, wherein that it provides for executing the diffusion procedure after the passage of a given time after the previous treatment procedure.

13. The execution method according to claim 1, wherein that it provides a measurement of at least one physico-chemical parameter of the air after a treatment procedure and, if a threshold is exceeded by the measured parameter, execution of an additional procedure of treatment of the air.

14. The execution method according to claim 1, wherein the gaseous composition comprises at least one volatilised compound, in particular for aromatherapy purposes.

15. The execution method according to claim 1, wherein it controls a light emitter in accordance with the session to be executed.

16. A system for a session of diffusion of an active gaseous composition in a habitable environment, the system comprising a device for diffusion of the gaseous composition, a device for measuring at least one physico-chemical parameter of the air and a device for treatment of the air, the system further comprising an application for controlling the devices that is arranged for the implementation of a method according to claim 1.

17. A system according to claim 16, wherein the diffusion device comprises at least one cartridge containing a formulation of the composition to be diffused that can be volatilised, and means for the volatilisation of the formulation in a flow of air that are controlled by the application.

18. A system according to claim 17, wherein it comprises a reader for reading information carried by the cartridge, the reader means being capable of transmitting the information to the application in order to control at least one characteristic of the diffusion on the basis of the information.

19. A system according to claim 17, wherein the cartridge is removable and/or resealable between two diffusion procedures (D).

20. A system according to claim 17, wherein the diffusion device comprises at least two capsules that can be used together or separately by the volatilisation means.

21. A system according to claim 17, wherein it further comprises a module dedicated to the control of the devices.
